(19) United States
(12) Patent Application Publication
(10) Pub. No.: US 2011/0257663 A1
(43) Pub. Date: Oct. 20, 2011

(54) SUTURE SYSTEM FOR MANUAL AND ROBOTIC SURGERY WITH SUTURE THREAD GATHERER AND FUSER AND DOUBLE POINTED SUTURE NEEDLE FOR ONE HAND APPLICATION (3)

(76) Inventor: John D. Unsworth, Hamilton (CA)

(21) Appl. No.: 12/737,489

(22) PCT Filed: Jul. 20, 2009
(86) PCT No.: PCT/CA2009/001022

§ 371 Date: Jul. 8, 2011

Related U.S. Application Data
(60) Provisional application No. 61/135,225, filed on Jul. 18, 2008.

(51) Int. Cl. A61B 17/04; A61B 17/06

(52) U.S. Cl. 606/144; 606/224

(57) ABSTRACT

The invention is a suture system including a device and a method for manual and robotic surgery with suture thread gatherer and fuser and double pointed suture needle for one hand suture application.

### BACKGROUND OF THE INVENTION

1. Field of the Invention

This invention relates to methods and devices to apply sutures remotely, robotically and manually.

2. Description of Prior Art

Sutures created with a suture needle and suture thread, have the advantage of evening out the forces acting on each point of attachment, by virtue of the suture or suture thread sliding through the needle formed hole and finding the lowest energy plateau along the suture or stitch length. This feature, along with the compliant nature of the suture thread minimizes trauma to the tissue that is joined. The other advantage of suture thread is that it can be made of biodegradable material, which is engineered to dissolve after the joined parts have grown together and the supporting strength of the thread is no longer required. This biodegradable aspect is sometimes not possible to engineer into metallic materials, which are often used in staples and clips.

The difficulty of using traditional sutures is that they require two hands or manipulators to apply them quickly. The needle must be inserted into the first surface, then across the surfaces to be joined and then removed as the tip of the needle protrudes above the second surface. The operator must then place another forceps on base or proximal end of the needle to initiate another stitch. Even in robotic surgery, the requirement for needle removal by the point (distal end) and reintroduction by the base (proximal end) requires two manipulators.

The other difficulty is that sutures must be tied at the beginning and end of the stitch line to secure it. Again this often requires two hands or manipulators to apply the correct tension and apply a secure knot. Another difficulty is that the application of the stitch and the tying of the knots require more space that is sometimes available, especially for endoscopic surgery. Another difficulty is that separate instruments or forceps types are required for the application of the suture and the tying and securing of knots.

What is needed is a suture needle which can be applied by one hand or one manipulator.

What is needed is a suture that can be securely joined together with one hand or one manipulator.

What is needed is suture method and system which can be effected in a confined space.

What is needed is a suture device which can both apply the suture needle and thread and then fuse together the threads in lieu of a knot.

### SUMMARY OF THE INVENTION

The invention is a suture system and method which allows for one handed suturing and knotting (fusing) of the suture thread within a confined space. The first aspect of the invention is a curved suture needle with double opposed points, as illustrated in FIG. 1 and 1a. As illustrated in FIGS. 6 to 6h, this needle can be inserted across the adjoining surfaces to be joined, pulled out, and then instead of the operator placing another forceps on the base or proximal end of the needle, the operator need only rotate the needle around its longitudinal axis, on that the opposite needle point can then be positioned, with the movement of the wrist, to pierce the surface for the next stitch. The stitching is accomplished by alternating needle ends for piercing, and rotating the curved needle around its longitudinal axis, between stitches, to eliminate the need for moving the forceps from the emerging tip of the suture needle to the base of the needle for the subsequent stitch. The double ended suture needle 1 also applies mattress stitches quickly, as illustrated in FIG. 2 and FIGS. 6i to 6ih, without passing the needle off to another forceps, again by alternating the ends of the needle for subsequent needle piecing operations.

The preferred embodiments of the invention include features which replace knotting of the suture thread with fusing it by heat or chemical means. The preferred embodiment is a suture applicator which includes a fusing pad at the distil end of a forceps, which holds two suture threads together securely, fuses them together with heat and pressure, and then releases them when the fused area cools sufficiently to be released. Alternatively, the fusing pad can dispense adhesives and/or solvents to chemically weld the suture threads together. These methods allow for the fusing of suture threads using only one hand or manipulator.

Suture thread may be referred to herein as simply thread, or visa versa. Threads also includes any flexible filamentous material, or thread-like material or line, including those materials which can be fused by heat or connected with adhesives or solvents, such as monofilament line and biodegradable suture thread.

While this disclosure refers to surgical sutures and suture threads, it is to be understood that these are only examples of a larger class of devices to which a thread is attached. For example fish hooks and lures can be attached to monofilament line using the fusing pad forceps herein disclosed, obviating the need for knots.

### BRIEF DESCRIPTION OF THE DRAWINGS

FIG. 1 is a perspective view of a double pointed suture needle 1 with suture thread 4. FIG. 1a is a perspective view of a double pointed suture needle 1 with suture thread 4 and terminal bead 4b.

FIG. 1b is a cross-sectional, perspective view detail of suture 1, suture thread 4 and terminal bead 4b, illustrated in FIG. 1a.

FIG. 1c is a cross-sectional, perspective view of a double pointed suture needle 1 with suture thread 4, connected terminal beads 4bi and 4bii, and extended hole 3b.

FIG. 1d is a cross-sectional perspective view detail of suture 1, suture thread 4 and terminal beads 4bi and 4bii, as illustrated in FIG. 1c.

FIG. 2 is a perspective view of a mattress stitch suture.

FIG. 3 is a perspective view of a conventional helical suture stitch.

FIG. 4. is a perspective view of scissor like forceps 7 which have various means for causing their clamping ends 9, 9a to move with respect to each other to impart a rotary motion on that which is clamped between them

FIG. 4a is a perspective view of a detail of the sliding connection 10a, joining the two proximal arms 12, 12a, allowing them to rotate and slide back and forth 10d, with respect to each other.

FIGS. 4b and 4c are cross-sectional perspective views of a flexible and/or elastic bushing 10e and deformed busing 10f, respectively, connecting the two proximal arms 12, 12a, at the joint 10.

FIGS. 4d and 4e are perspective views of a detail of the distal end of the forceps 7 (which may or may not have a compliant joint 10) which in this preferred embodiment includes a curved, springy distal arm 11, which can remain relatively rigid when the forceps grasp an object between the clamping ends 9, 9a, but when sufficient force is applied 11c, flexes and flattens the arc 11b of springy distal arm 11, causing the clamping end 9 to extend in direction 11d, and causing an object, in this case a suture needle 1 to rotate 1a.

FIG. 5 is a perspective view of forceps 7, which includes a suture thread fusing pad 13 on the contact surface of clamping end 9 and fusing energy/chemical conduit 13a.

FIG. 5a is a cross-sectional perspective detail view of the distal end of the forceps 7 which illustrates the suture threads 4 which are to be fused, held in recesses 20, adjoining thread guides 15.

FIG. 5b and 5c are a cross-sectional perspective detail views of the clamping end 9, along line 14 shown in FIG. 5a, illustrating the fusing pad 13 and compressible restraining pad(s) 16. The recesses 20 are not shown on this detail to illustrate another embodiment.

FIG. 5d is a perspective overhead view of the distal end of clamping end 9, with round fusing pad 13 and round compressible restraining pad 16, and suture thread guides 15.

FIGS. 5e to 5h are perspective, cross-sectional views of a sequence of steps illustrating a springy clamping end 9a in combination with a projection 9b which can grip thread 4, so that another thread may then be gathered together with the first thread, so they may be fused together.

FIGS. 5i and 5j are perspective, cross-sectional views illustrating how the springy clamping end 9a may be directly controlled by a finger hole 8c and draw wire 8f.

FIG. 6 to 6h are cross-sectional perspective views of a typical helical stitch procedure in which opposite suture needle 1 points are used to pierce the body 5a for each successive stitch from FIG. 6 through FIG. 6h.

FIG. 6i to 6ih are cross-sectional perspective views of a mattress stitch procedure in which opposite suture needle 1 points are used to pierce the body 5a for each successive stitch from FIG. 6i through FIG. 6ih.

FIGS. 7 and 7a are perspective views of tweezers type forceps 7b having a distal end the same as that illustrated in detail in FIGS. 4d and 4e, with distal arm 11 being curved and springy.

FIGS. 8, 8a, 8b, 8c and 8d are perspective views of tweezers type forceps 7b that have an elastic 10e or integral spring type member 10ei, which allows the operator to vary the relative direction of movement of clamping ends 9 and 9a along various axes.

FIGS. 9 and 9a are perspective views of tweezers type forceps 7b that has a groove 20, which forms a journal in which the suture needle 1 can rotate, rather than rolling down the clamping end 9a when a rotary motion 1a is imparted by the operator creating relative motion 11d between the clamping ends 9, 9a.

FIGS. 10 and 10a are perspective views of tweezers type forceps 7c that can act as a suture needle manipulator, as that illustrated in FIGS. 9 and 9a, but also act as suture thread fuser with functions similar to that illustrated in FIG. 5a.

FIGS. 10b, 10c and 10d are perspective cross-sectional views of forceps 7c having a curved clamping end 9, and recess 20 which can fuse suture threads 4 together, on recessed fusing pad 13, as illustrated in FIGS. 10b, and 10c, or manipulate objects such as a suture needle 1 within the grasp of clamping ends 9 and 9a and on recess 20, to aid in rotating objects, as illustrated in FIG. 10d.

FIGS. 11 and 11a are perspective views of tweezers type forceps 7b as illustrated in FIGS. 9 and 9a, except the operator has moved distal arm 11 laterally in direction 11e, as illustrated in FIG. 11a.

FIGS. 12 and 12a are perspective cross-sectional views of the distal end of the clamping ends 9, 9a and illustrate how a suture needle 1 can be grasped at the distal end and rotated la by moving the clamping ends laterally with respect to one another as illustrated in FIGS. 11 and 11a.

FIGS. 13 and 13a are perspective cross-sectional views of the distal end of the clamping ends 9, 9a this have mating surfaces that are on the bias and a springy clamping end 9.

FIGS. 14, 14a and 14b are perspective, cross-sectional views of a robotic end-effector 7d, which is has only one wire to control suture needle 4 holding and rotation.

FIG. 15 is a perspective, cross-sectional view of a robotic or remotely controlled tweezer type, end-effector 7e, which has two wires to control suture needle 1, holding and rotating it, as well as acting as a suture thread 4 fuser.

FIG. 16 is a perspective, cross-sectional view of a robotic or remotely controlled end-effector 7e which has a single wire or actuator to control suture needle 1 holding and rotating it.

### DETAILED DESCRIPTION OF THE INVENTION

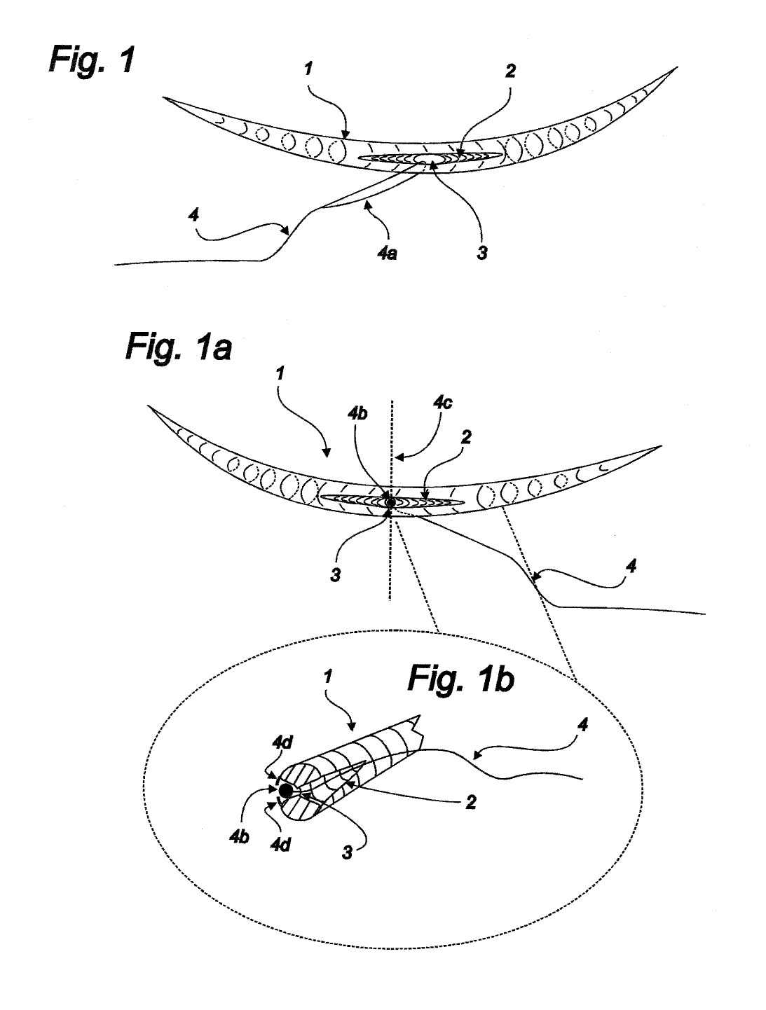

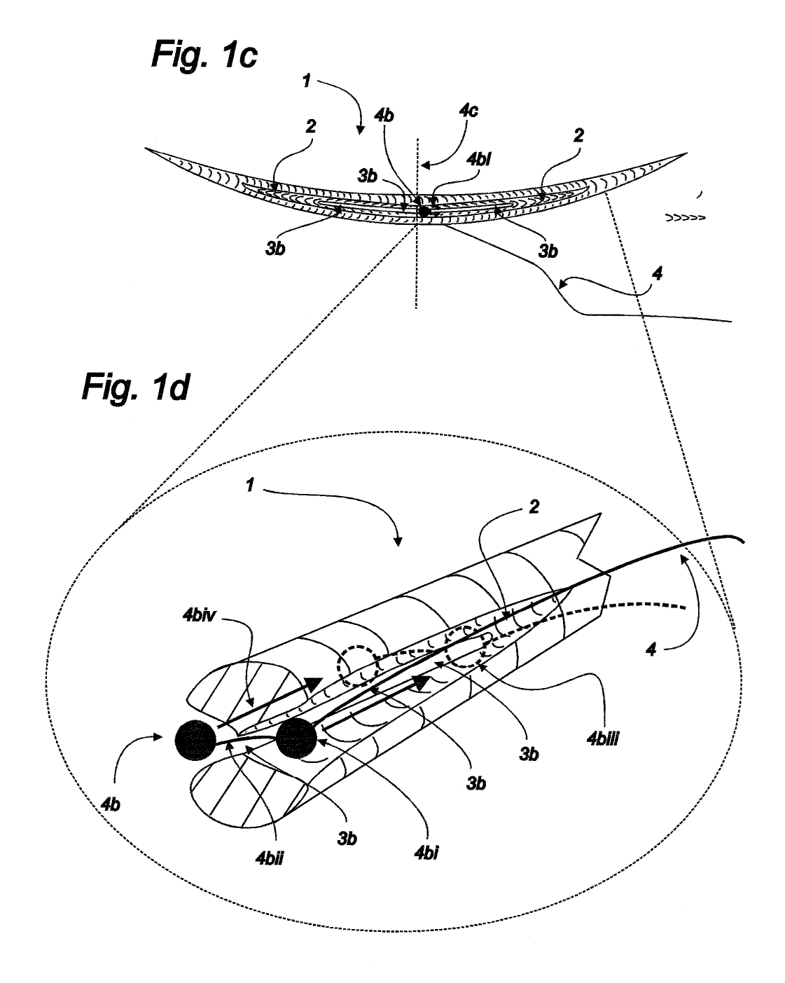

FIG. 1 illustrates a double pointed suture needle 1 with which has a hole 3 to accommodate the suture thread 4. in some embodiments of the invention, the said hole walls are flared out 2 on one or both sides of the needle to accommodate the thread 4 and thereby minimize trauma to the tissue, as the suture needle and thread are pushed into the body. In this embodiment of the invention the suture thread 4 is formed into a loop 4a which secures it to the suture needle. Some preferred embodiments of the invention do not use a loop 4a but rather connect the suture thread 4 to the suture needle 1 using a swage, crimp, adhesive or by connecting it by other means well know to the art. In some preferred embodiments, illustrated in FIG. 1a and detail FIG. 1b along line 4c, rather than crimping the end of the thread 4 into a hole 3, have a bead 4b formed into the end of the thread 4 or attached to it. In these embodiments of the invention, this bead or enlargement 4b secures the thread 4 to the needle 1, on one side of the needle 1. The said thread 4 passes through the hole 3, which is smaller than the bead 4b, to the other side of the needle 1. This loose footed bead 4b allows the thread to move at various angles with respect to the needle 1, and reduces the radius of bends that would otherwise occur, when the thread 4 is simply crimped into a hole 3 in the needle 1. By reducing the radius of the bend at the thread 4, needle 1 interface, trauma to the tissue is reduced. In one embodiment, an elastic bead 4b may be pressed into a retaining socket, (a hole 3 with a closed or partly closed bottom) and then allowed to expand, so as not to fall out, but remain loose and mobile within the socket. In other embodiments of the invention the bead may be fitted into a hole 3 and a rim or protrusion 4d may be formed after to form the socket, and thus retain the bead, as illustrated in FIG. 1b. In other embodiments of the invention the thread 4 is attached to the needle 1 with adhesive, or is an extension of the material comprising part or all of the needle 1. Another advantage of this embodiment is that the loose footed bead 4b allows the thread 4 to rotate about its longitudinal axis and thereby relieve any tangling that might occur.

FIG. 1b and 1c a detail view of FIG. 1b, is another embodiment of the invention in which the hole 3b is extended in both directions to permit the beads 4b and 4bi slide along 4biv (as illustrated in FIG. 1c) the extended hole 3b to position 4biii, to prevent interference with the grasping forceps on each stitch. Beads 4b and 4bi can be connected by connecting thread 4bii, or other connecting means well known to the art. Some embodiments of the invention utilize other more streamlined shapes for the beads, or contour them out of one piece, to minimize trauma on the tissue, and the shape of these will depend upon the use to which the invention is put, and all come within the ambit of the invention. Other embodiments of the invention may use other connecting means in association with the suture needle having an extended hole 3b, such as a loop 4a illustrated in FIG. 1, and all are within the ambit of the invention.

FIG. 1c is a cross-sectional, perspective view of a double pointed suture needle 1 with suture thread 4, connected terminal beads 4bi and 4bii, and extended hole 3b.

The dimensions and shape of the suture needle 1 will vary depending upon the particular application, and all are within the ambit of the invention. The principle advantage of the double pointed needle is its ability to be applied singlehandedly. This is due to two features, the first is the double opposed point and the second is the curve of the needle, which when rotated about its longitudinal axis reduces the amount of wrist action the surgeon is required to make on subsequent stitches, especially helical stitches. Some embodiments of the invention apply a fine texture to the surface of the suture needle 1, at a location where the forceps makes contact to improve the grip, by means well known to the art. This improves the forceps grip on the suture needle 1 and makes rotation of the suture surer, by those embodiments of the invention described below. Other preferred embodiments include flat plateaus in the otherwise rounded surfaces of the suture needle, at locations where the forceps can have a preferred location relative to the suture needle 1, so as to more predictably position the suture needle with respect to the forceps position.

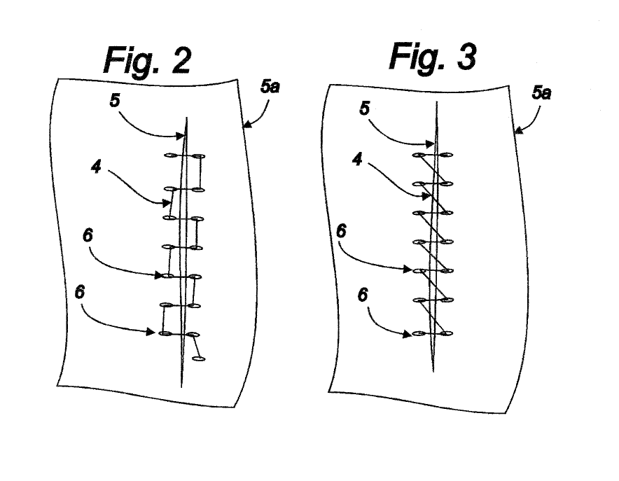

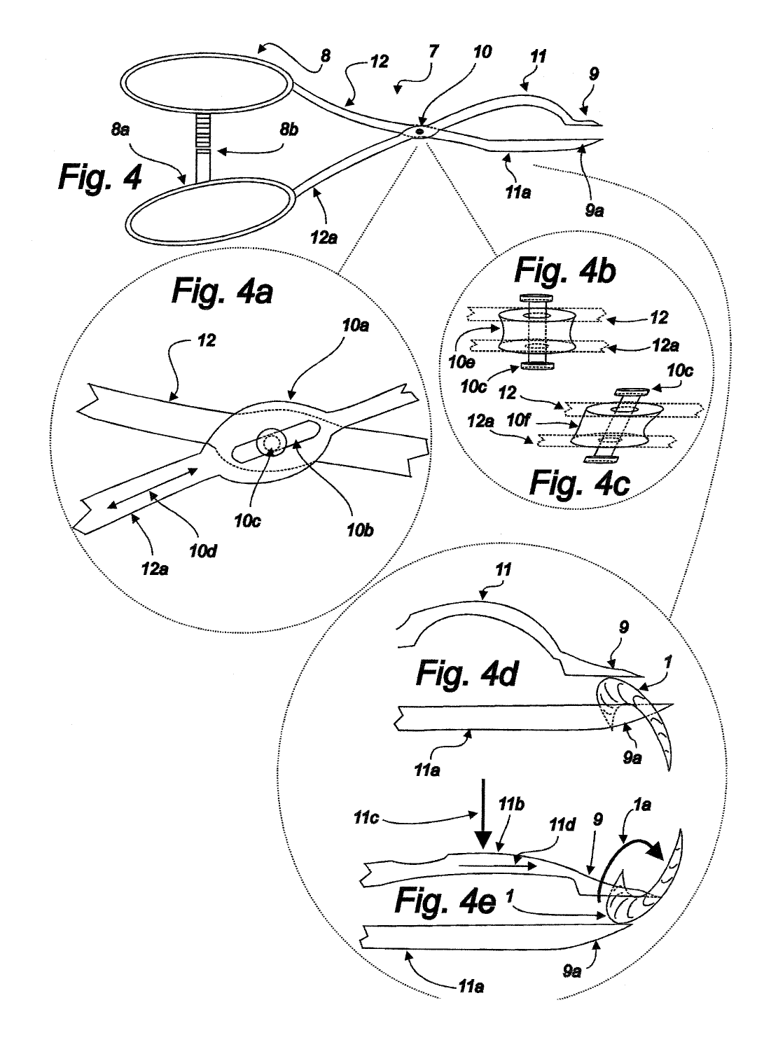

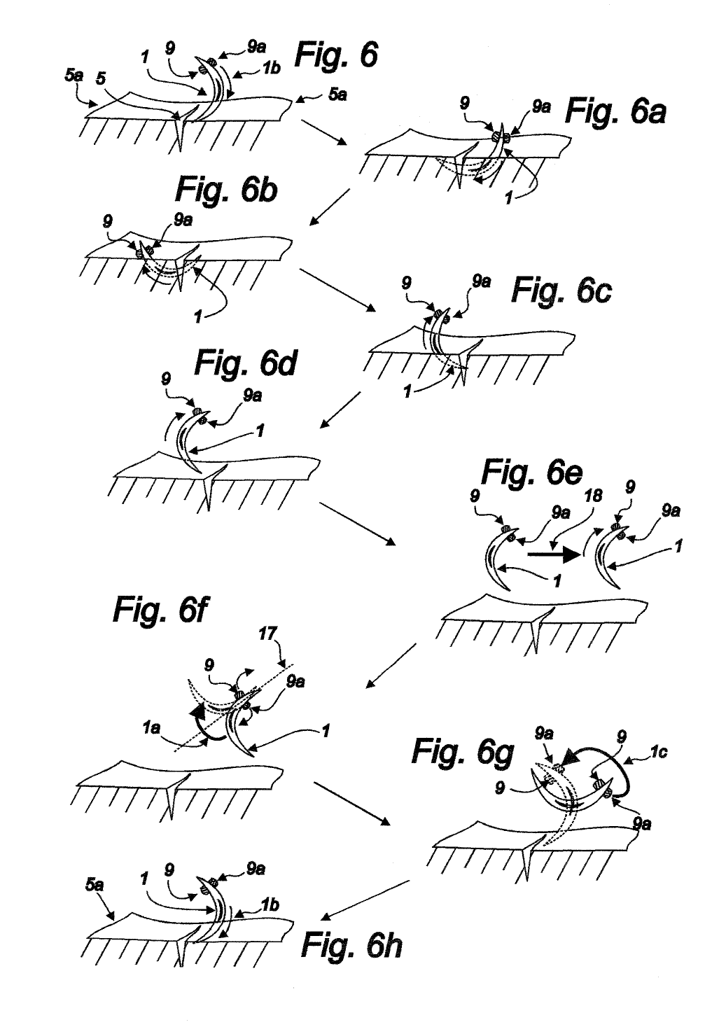

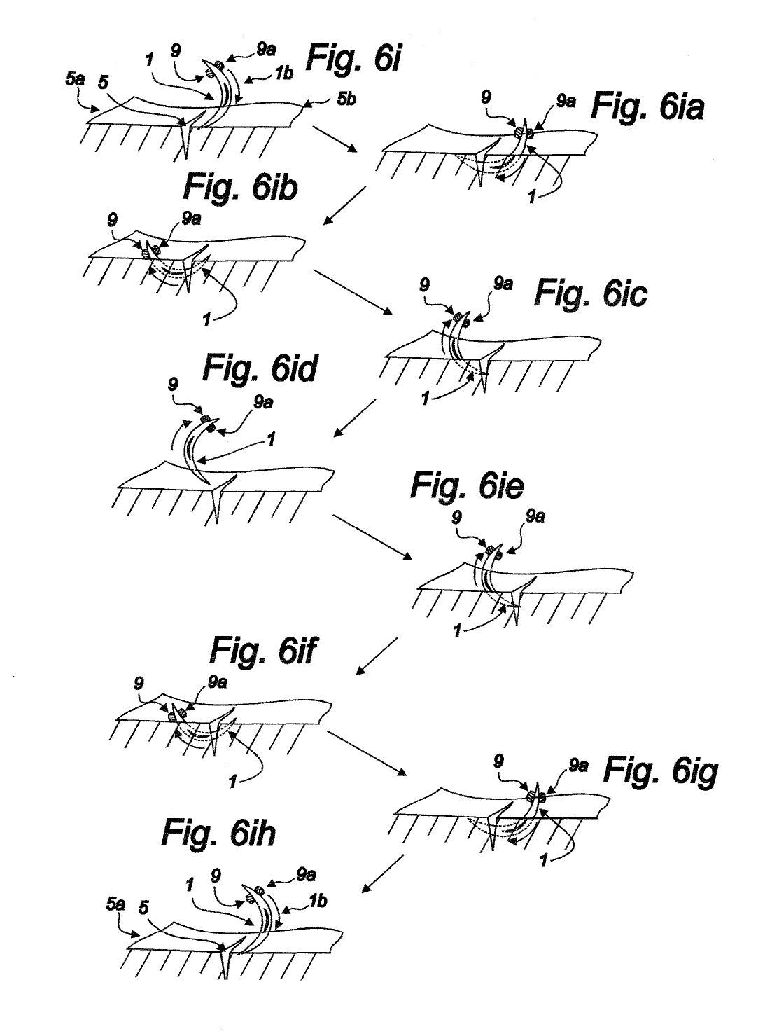

The advantage of the double pointed suture needle 1 is readily appreciated when considering the mattress stitch, which is illustrated in FIG. 2, and the forming of which is illustrated in FIGS. 6i to 6ih. In FIG. 2 an incision 5 in body 5a is being closed with suture thread 4 which has been threaded through suture holes 6 to form the mattress stitch. The details of the forceps motion, and hence wrist motion of the surgeon, required to apply the stitch are illustrated in FIGS. 6i to 6ih. FIGS. 6i to 6th show the clamping ends 9, 9a of forceps 7, such as those illustrated on FIG. 4, and from these the position of the surgeon's wrists can be deduced. As these drawings illustrate the suture is first applied by inserting the suture needle 1 in a curved motion 1b as shown on FIG. 6i. The suture needle 1 can be withdrawn from the body by grasping the protruding point, as illustrated in FIG. 6ib 6ic and 6id, and then immediately, without handing off the suture needle 1 to the other hand, immediately reintroduce the suture needle 1 into the body, using the opposite point, as illustrated in FIG. 6ie. The stitch line is thus singlehandedly produced by alternating the piercing point on each stitch. There is no need to move the forceps from the pointed end or distal end of the needle, when the needle protrudes above the surface of the body, to the base or proximal end, to initiate another stitch by piercing the body. Hence, there is no need to have a second forceps to make the exchange from gripping the distal end to the proximal end.

The advantages of the double pointed suture needle 1, also applies to the helical stitch, which is illustrated in FIG. 3, but is best used with special forceps, which are able to rotate the suture needle 1 about the needle's longitudinal axis, and which form part of the invention.

The helical stitch is the most commonly used stitch, so it is important that these stitches can be accomplished singlehandedly or by one manipulator of a robot. The details of the forceps motion, and hence the wrist motion of the surgeon, required to apply the stitch are illustrated in FIG. 6 to 6h. For diagrammatical clarity, the thread and FIGS. 6 to 6h show the clamping ends 9, 9a of forceps 7, such as those illustrated on FIG. 4, and from these the position of the surgeon's wrists can be deduced. FIGS. 6 to 6d illustrates the first stitch being applied. Using a single point suture needle, at this point, the surgeon would hand-off the suture needle to his other hand, so that he can move the position of the forceps from the point of the needle, the distal end, to the base of the needle or proximal end, for reintroduction of the suture needle into the body for the next stitch. The hand-off also allows the surgeon to divide the required rotation of the needle between both wrists.

However, using the double point suture needle 1, the next stitch, starts with moving the suture needle across 18 the incision, as illustrated in FIG. 6e. Next the suture needle 1 can be reintroduced into the body 5a for the next stitch, singlehandedly by breaking the change in needle attitude into two steps. The first step is illustrated in FIG. 6f, where the suture needle 1 is rotated 1a approximately 180 degrees about its longitudinal axis 17. This rotation turns the point up as illustrated in FIG. 6f and by doing so, the subsequent wrist rotation 1c can reduced to approximately 90 degrees, as illustrated in FIG. 6g. The suture needle 1 is now in a position to be reintroduced into the body to form the next stitch. Note that on each subsequent stitch, the opposite point of the suture needle 1 is used to introduce the needle into the body with a curved motion 1b, so that the surgeon need not reposition the forceps from the pointed end to the base end, for each stitch. Obviously the two steps described above may be executed in reverse order and in some cases, where the position of the body relative to the forceps varies or is appropriate, only one of the two steps may be required. Also for diagrammatical clarity, the thread 4, which is connected to the needle 1 is not shown.

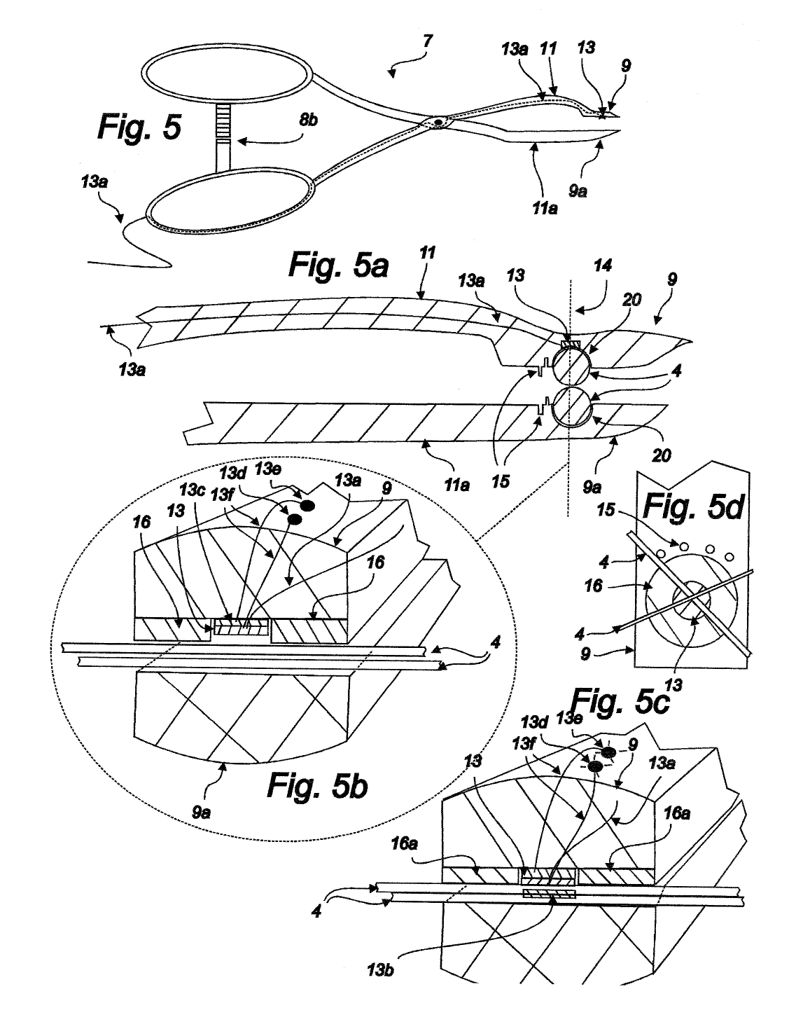

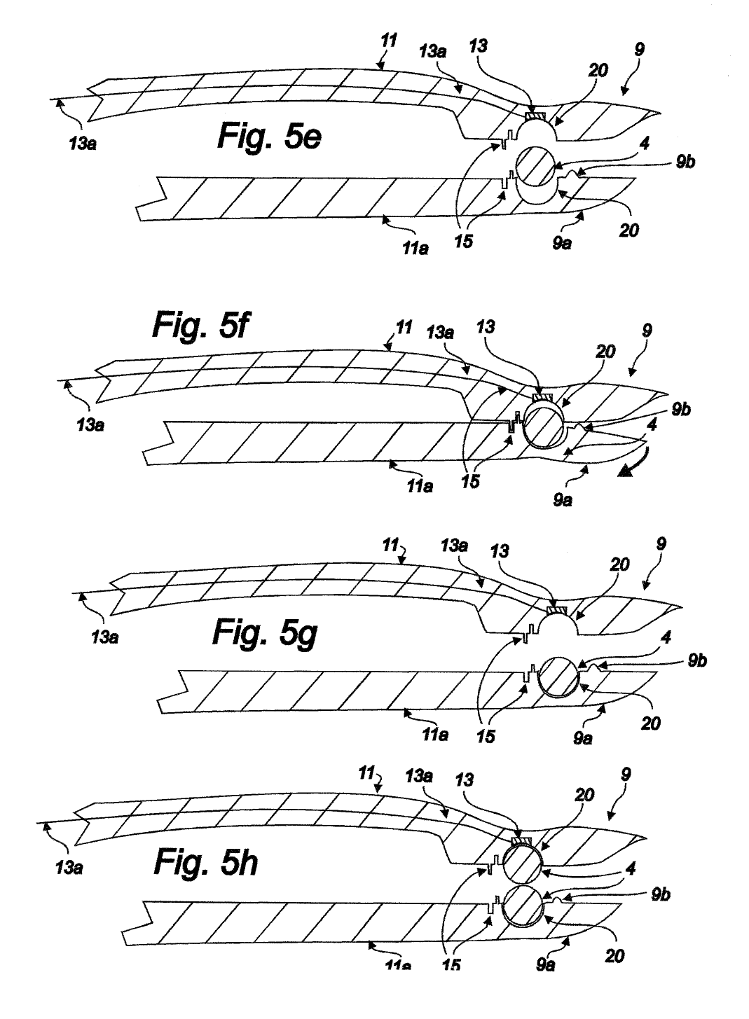

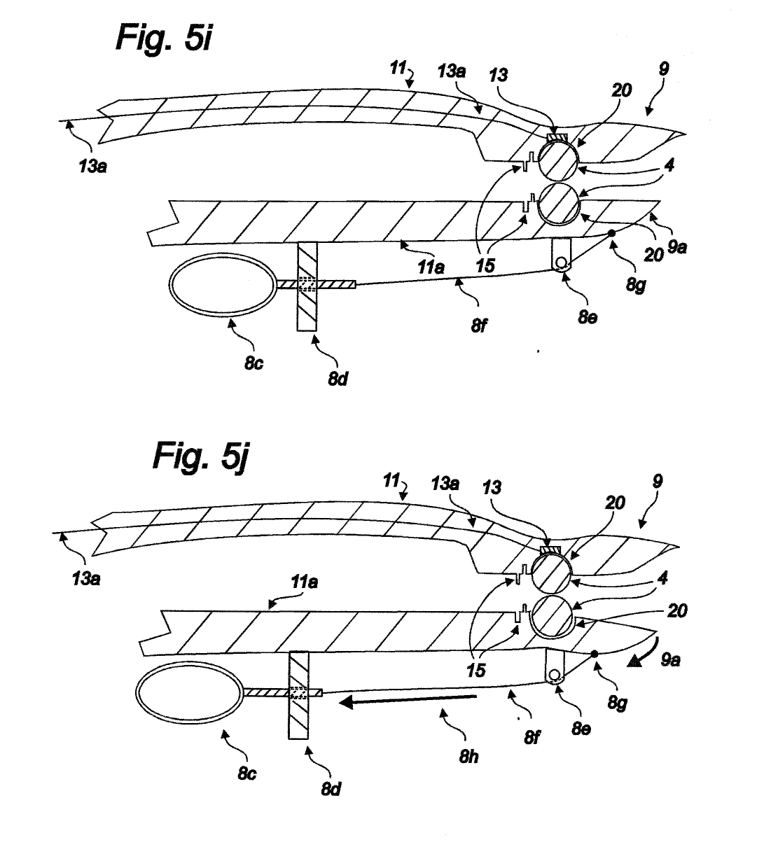

As mentioned above, a method of rotating 1a the double pointed suture needle 1, approximately 180 degrees around its longitudinal axis 17 requires a forceps that can hold, rotate and release the forceps, using one hand. The preferred embodiment of the invention includes the combination of the double pointed suture needle 1 and needle rotating forceps, such as those illustrated in FIGS. 5, 7, and 8. The needle rotating forceps forming part of the invention create the rotation by altering the relative motion of their clamping arms 9, 9a.

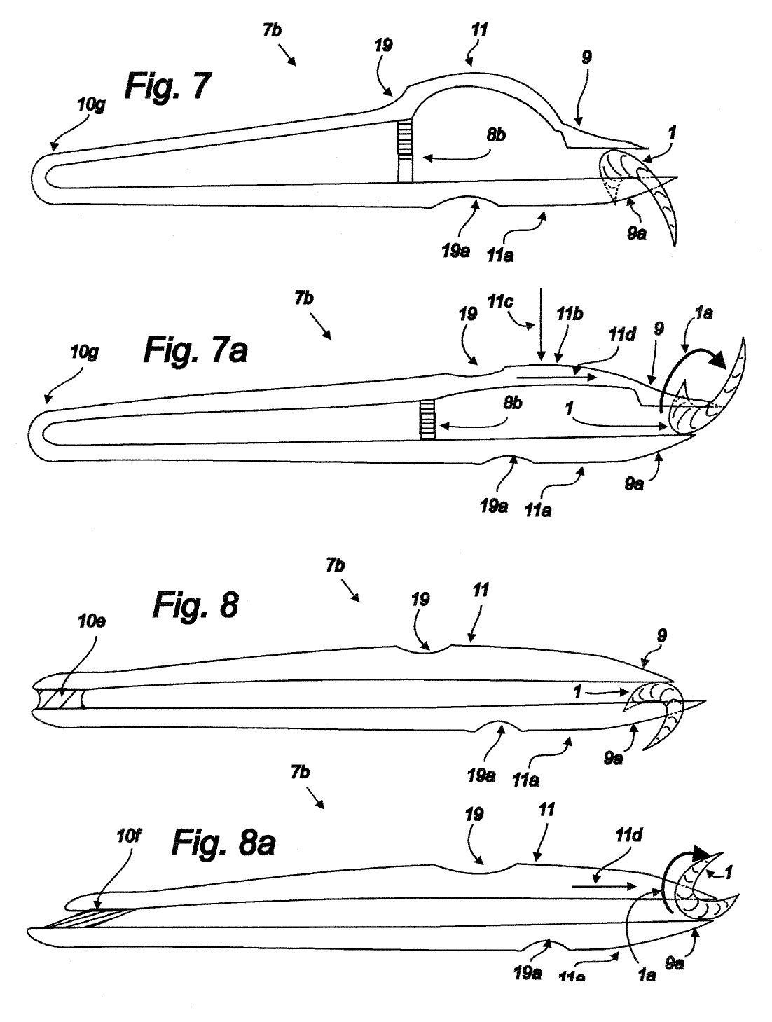

FIG. 4. is a perspective view of scissor like forceps 7 which have various means for causing their clamping ends 9, 9a to move with respect to each other to impart a rotary motion la on a suture needle 1 or on that which is clamped between them. While there will be many other uses for forceps which can impart a rotary motion on the object grasped by them, and all these are within the ambit of the invention, of particular relevance is their use to rotate the double pointed suture needle 1, to change the suture attitude between successive stitches as described above, and illustrated in FIGS. 6 to 6h. FIG. 4 also illustrates a ratchet 8b, that is well known to the art and which allows the forceps to be closed and latched into the desired position, so the operator need not continue to press the forceps finger holes together, while holding an object between the clamping ends 9, 9a. These ratcheting 8b means may take many forms well known to the art and may be included in the pincer type of forceps such as those illustrated in FIGS. 7 and 7a. These ratcheting means also may be released by a simple lateral deflection of the relative position of the forceps finger holes 8, 8a or distal arms 11 11a, or by simply increasing the closing pressure, all of which are well known to the art. Although the ratcheting means are not shown on all the illustrations contained herein, it is to be understood that they may be included in any an all of the embodiments of this invention and may be located in any convenient part of the forceps, and all are within the ambit of the invention.

FIG. 4a illustrates one embodiment of the invention which allows for relative motion between the clamping ends, 9, 9a, is a sliding joint 10a, which has a channel 10b which allows the proximal arms 12, 12a of the forceps to slide back and forth in directions 10d, while the retainer pin 10c maintains the sliding connection between the forceps proximal arms 12 and 12a. As can be readily appreciated this motion is translated into a relative motion of the distal arms 11, 11a and the clamping ends 9, 9a. This relative sliding motion can then be created by the operator simply moving the fingers and thumb, back and forth, in the finger holes 8 and 8a, as illustrated in FIG. 4

Other preferred embodiments of the invention use other methods of allowing for the relative motion between the forceps proximal arms 12 and 12a and/or the clamping ends 9, 9a. FIG. 4b illustrates how an elastic bushing 10e between offset proximal arms 12 and 12a can allow for similar relative motion between the arms, caused by the movement of the operator's fingers and thumb in the finger holes 8 and 8a. The bushing can distort 10f in response to operator inputs, as illustrated in FIG. 4c. The advantage of this system is that the motions imparted are not just back and forth motions 10d as illustrated in FIG. 4a, but can be relative sliding motions along any axis in the plane parallel and between the clamping ends 9, 9a, for example side to side, on a bias or even rotary. The operator can then manipulate the suture needle 1, in a more dynamic way. While FIGS. 4b and 4c include a retainer pin 10c, other preferred embodiments do not include such a connector, but connect the bushing to the forceps proximal arms 12 and 12a with adhesive or other connecting means, relying on the bushing to torque sufficiently to accommodate the opening and closing motion of the said forceps proximal arms 12 and 12a.

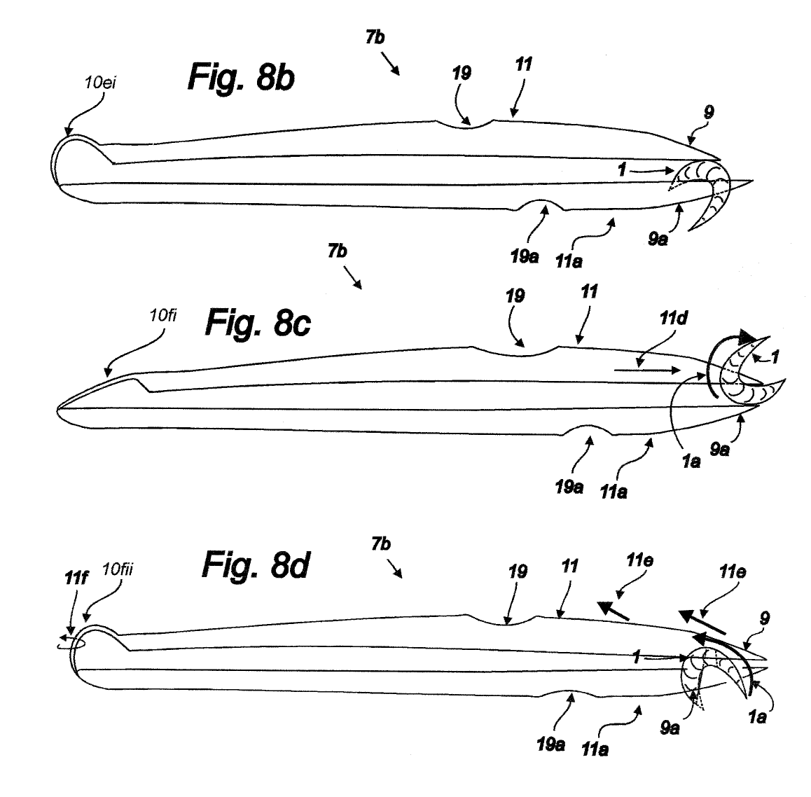

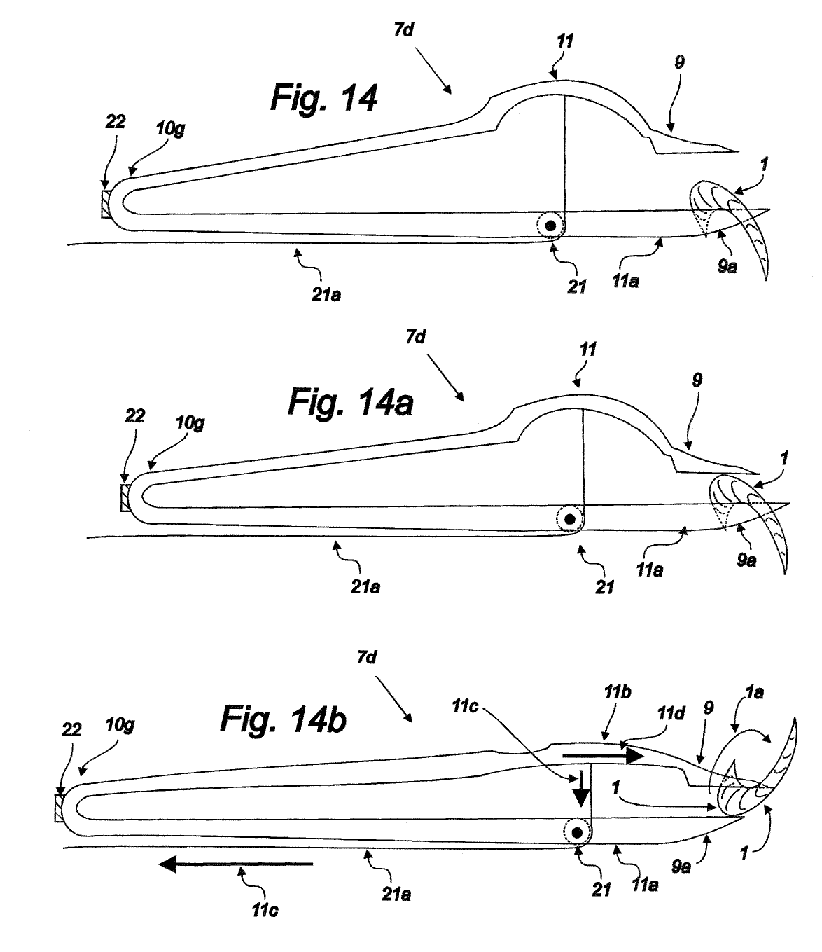

Other preferred embodiments create the relative sliding motion between clamping ends 9, 9a by having a rigid distal arm 11a and a curved, springy distal aim 11 as illustrated in FIGS. 4d and 4e. The flexible distal arm 11 can remain relatively rigid when the forceps grasp an object between the clamping ends 9, 9a, as illustrated in FIG. 4d, but when sufficient additional force is applied 11c, as illustrated in FIG. 4e, the curved, flexible distal arm flexes 11b, causing the flexible distal arm to extend in direction 11d, and causing an object, in this case a suture needle 1 to rotate 1a. This embodiment of the invention has the advantage of not requiring an additional arm or actuator to impart rotary motion on the suture needle 1. The downward force applied is simply increased above a certain threshold, beyond which the clamping force is accompanied with a sliding motion, which can impart the desired motion to the object being held between the clamping ends 9, 9a. This embodiment of the invention is especially suitable for robotic or endoscopic surgery, where a single wire or actuator can provide both clamping and rotary motions, and also where space is limited. The other advantage of this embodiment is that an approximate, ideal clamping force can be maintained while the sliding force is applied. This flexible distal arm 11 and rigid distal arm 11a combination may be part of scissor type forceps, tweezers type forceps, robotic forceps or other embodiments of the invention. FIGS. 7 and 7a illustrate this system as a tweezers type forceps and FIGS. 14 and 14a as a robotic type forceps or end-effector. In these embodiments of the invention, a springing element 10g maintains the distance between the clamping ends 9, 9a, until downward force is applied, although one can appreciate that any suitable spring or bushing 10e, as illustrated in FIG. 8, or springy member 10ei illustrated in FIG. 8b could be substituted, or any similar energy storage device, well known to the art, and all within the ambit of the invention.

The robotic end-effector illustrated in FIG. 14, which is connected to the robot arm with interface 22, illustrates how one wire 21a, pulled around turning guide 21, in combination with a springing element 10g can cause the clamping ends which start in an open, unloaded condition. FIG. 14 to clamp down and hold the suture needle 1, FIG. 14a. FIG. 14b illustrates that upon exerting additional force 11c, the semi-rigid distal arm 11 (illustrated in FIG. 14a) deflects 11b, causing it to extend longitudinally 11d, and thus causing the suture needle 1 to rotate 1a. When the wire 21a is slackened, the loaded springing element 10g unloads causing the end-effector 7d to return to its confirmation illustrated in FIG. 14.

While the mating surface of the clamping arms 9a are horizontal, as illustrated in FIGS. 7 and 7a, which results in the longitudinal relative motion 11d, they could also be biased, as illustrated in FIG. 13 and FIG. 13a, when in combination with a horizontally springy clamping ends 9, or 9a, or both, a downward force 11c effected by the operator, results in a sliding, lateral relative motion 11g. Thus, changes in the relative geometry, materials and design of the clamping arms, all well known to the art can cause many different trajectories of the clamping ends 9, 9a, and thus many different imparted motions of the suture needle 1 being held in their grasp. All these preferred embodiments are within the ambit of the invention.

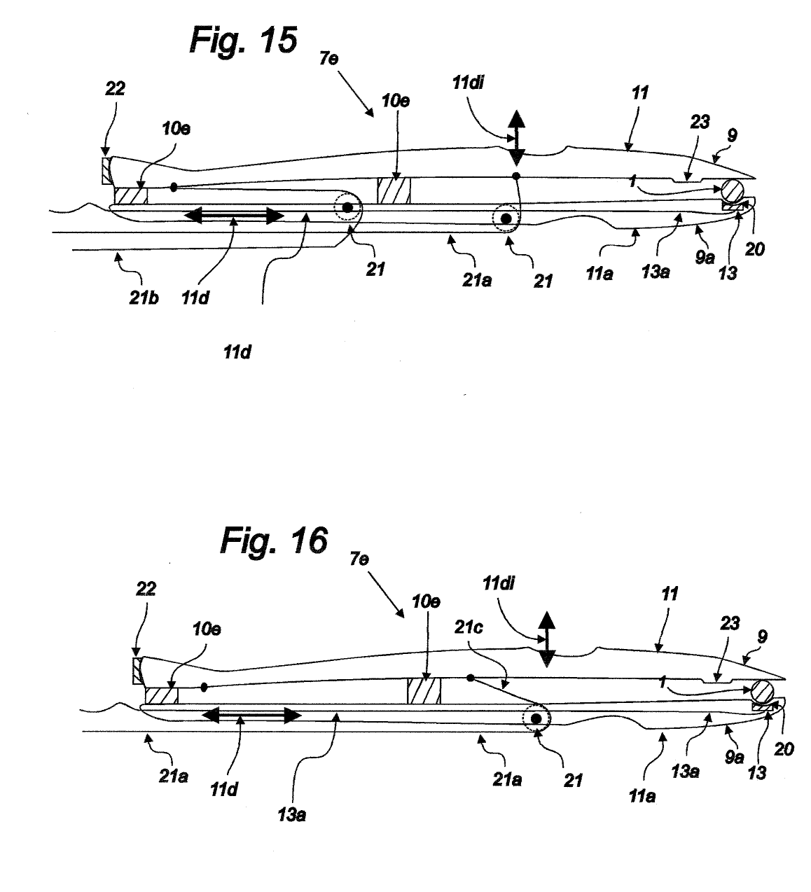

The robotic end-effector 7e illustrated in FIG. 15, which is connected to the robot arm with interface 22, illustrates how two wires 21a and 21b, with springy element 10e can control both the relative vertical positions 11di of distal arms 11 and 11a, of a tweezer type of forceps, by pulling and releasing wire 21a, and also the longitudinal motion 11d by pulling and releasing wire 21b around turning guide 21. This end-effector can act both as an object manipulator, but a suture fuser as well.

FIG. 16 illustrates how a single wire 21a at approximately at approximately 45 degrees from vertical 21c, can simultaneously effect a relative vertical motion 11di and a relative longitudinal motion 11d of distal arm 11a.

It is to be understood that the relative motions may be effected by other types of actuators, and motion transmission devices, set at different angles, well known to the art, and that wires 21a and 21b are intended to be examples of all these other means, and all are within the ambit of the invention.

FIGS. 8 and 8a are perspective views of embodiments of the invention in the form of tweezers type forceps 7b that have an elastic or spring type member 8e, which allows the operator to vary the relative direction of movement of clamping ends 9 and 9a along various axes, not just back and forth, as is the case for that embodiment illustrated in FIG. 4a. FIG. 8a illustrates the effect of the operator pushing the upper arm 11 from left to right 11d, distorting the elastic member 10e, illustrated in FIG. 8, into distorted form 10f, illustrated in FIG. 8a, and imparting a rotary motion on the suture needle 1 which is held by clamping ends 9, 9a as illustrated in FIG. 8a. This embodiment of the invention includes a thumb recess 19 and finger recess 19a, to make their use more comfortable, but other finger rings or other finger grips, well known to the art, may be used in other embodiments of the invention.

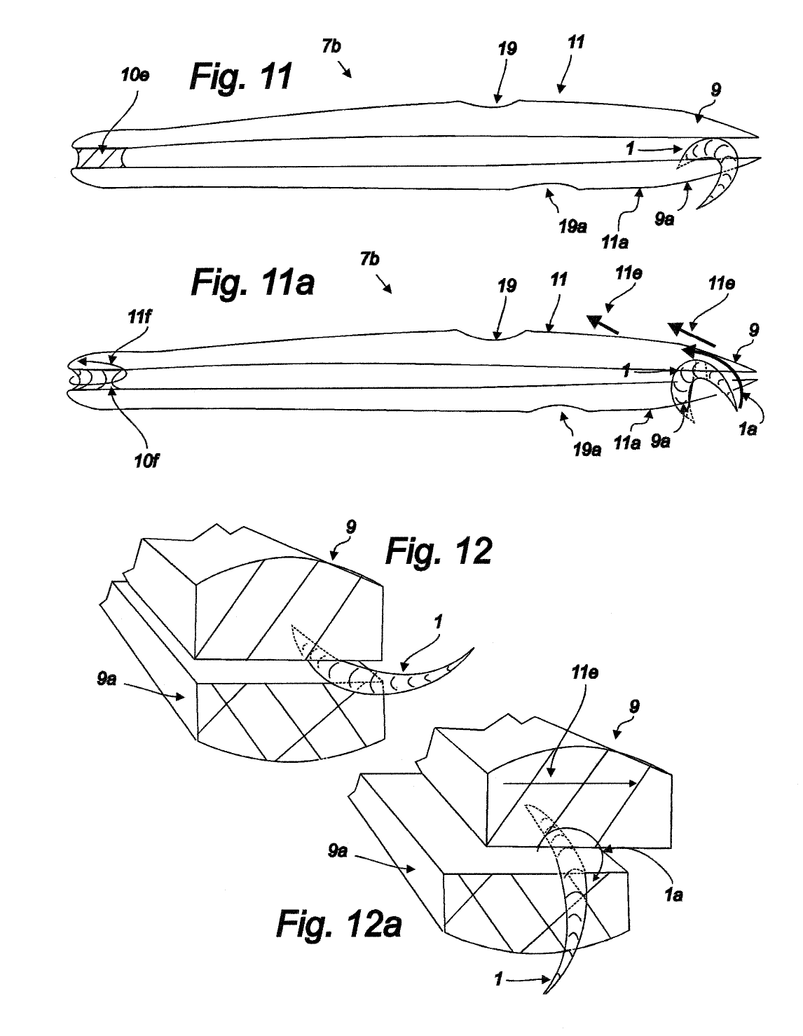

FIG. 8b 8c and 8d illustrate an integral spring member 10ei, which can be connected to the upper distal arm 11 and lower distal arm 11a, or can be formed from the said arms into a one piece element. FIG. 8c illustrates the effect of an operator pushing the upper distal arm 11 from left to right 11d, distorting the spring member 10ei, as illustrated in FIG. 8b, into distorted form 10fi, in a similar fashion to that illustrated in FIGS. 8 and 8a. FIG. 8d illustrates how the spring element 10ei, as illustrated in FIG. 8b, can be distorted in radial direction 11f, by the operator moving the upper and lower distal arms 11, 11a laterally with respect to another, in a similar manner as illustrated in FIGS. 11 and 11a. FIG. 8d illustrates the upper distal arm 11 being moved in direction 11e by the operator. Obviously the operator may combine lateral and back and forth movements to rotate the suture 1 in various directions.

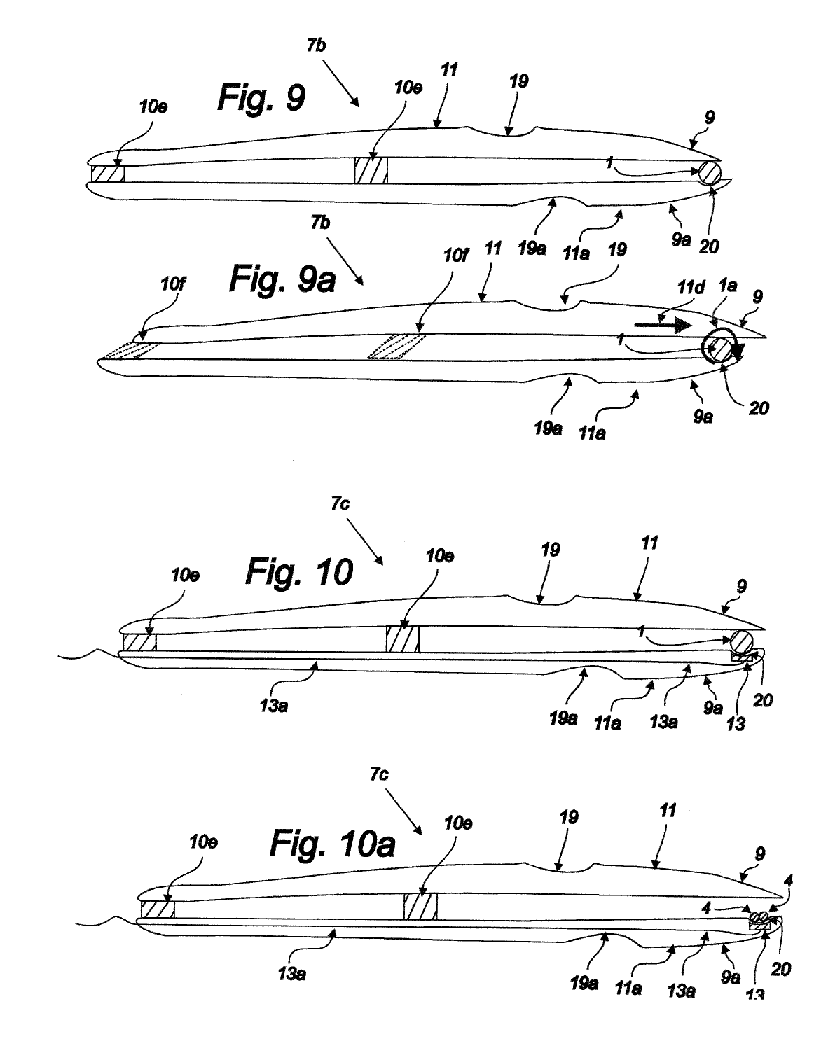

FIGS. 9 and 9a illustrate a similar forceps 7b arrangement, but a recess 20 has been added to the clamping end 9a so that when the upper distal arm 11 is moved longitudinally 11d, the recess 20 provides a journal in which the suture needle 1 can rotate, without the needle walking down the surface of clamping end 9a. This journal 20 may of course be applied to any of the embodiments of the invention and may be located on either distal arm 11, 11a or both. It is important to note that the suture needle 1 may be grasped at other points along the surface of clamping ends 9, 9a and be manipulated without recourse to the journal, if the operator finds this more convenient.

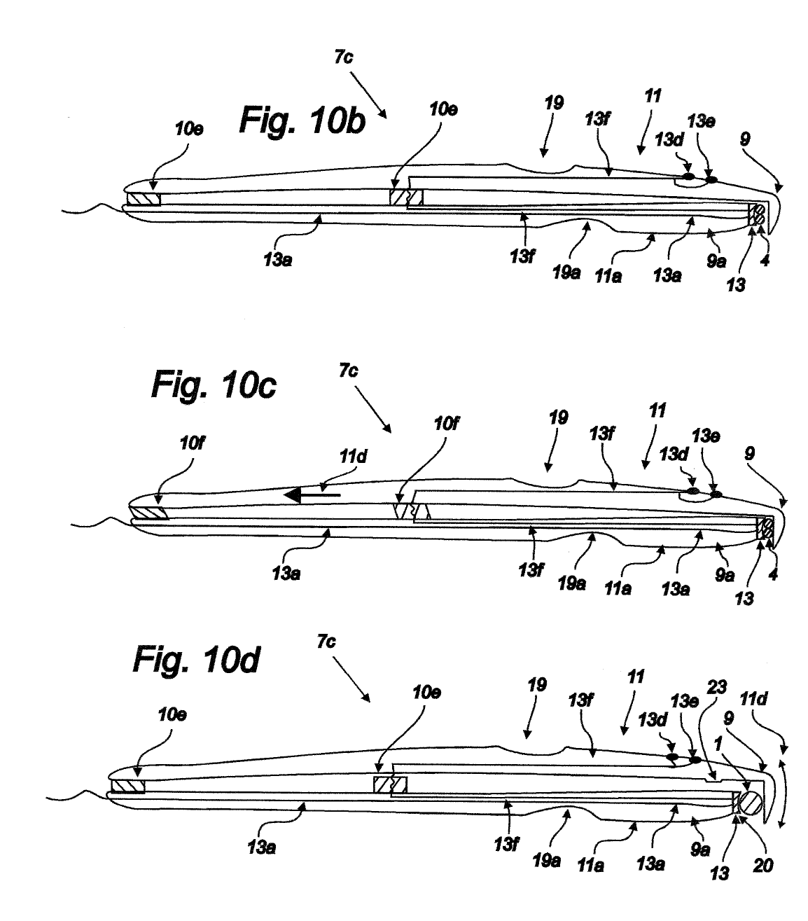

FIGS. 10 and 10a illustrate how this journal 20 may be used to manipulate suture needles as illustrated in FIGS. 9 and 9a, but also may include a fusing pad 13 to heat or chemically fuse the suture thread 4 together. In this embodiment of the invention, the threads 4 are gathered and fused side be side.

FIGS. 5, 5a, 5b, 5c and 5d illustrate in greater detail the method of fusing by heat the suture threads 4, in place of knotting, which can be difficult in confined spaces and difficult for robots to do. The clamping ends 9, 9a can include a fusing pad 13 to which energy is delivered by energy/ chemical conduit 13a. Some embodiments of the invention use a fusing pad which is a resistive heating element, to which electric energy is delivered along energy/chemical conduit 13a, from a remote source. This heat is sufficient to melt or partly melt all or some of the polymer, plastic or other fusible thread 4, causing the threads to fuse together. When the heating is terminated by turning off the energy, the threads solidify together forming a welded bond. Some embodiments of the invention have foot switches or other switches and timers well known to the art to regulate the amount of energy that is supplied to the fusing pad 13. Other preferred embodiments use pressure switches 13c that are integral to the fusing pad, as illustrated in FIG. 5b, turning on the current, when a predetermined pressure is exerted on the pad, and turning it off, when the pressure drops below a predetermined threshold. This would allow the operator to first collect the suture threads together against guides 15, as illustrated in FIG. 5a, and then closing down on the threads with the clamping ends 9, 9a, as illustrated on FIG. 5b. At this point the compressible restraining pads 16 engage the suture threads 4 and secure them for fusing together. At this point, for this embodiment, the fusing pad 13 is not in contact with the suture threads 4, and only with the application of further pressure, as illustrated in FIG. 5c, does the fusing pad 15 conic in contact with the suture threads 4 and apply pressure to them. At this point the operator can switch on the heating energy, or the integral pressure switch 13c can turn on the energy at some predetermined pressure point. At this point the indicator lights may turn on, as indicated in FIG. 5c, indicating that the ideal fusing pressure has been achieved 13e, and that the power to the fusing pad 13 has turned on 13f. The suture threads 3 then weld together at the fused area 13b. At this point the operator can relieve some pressure allowing the fusing head to retreat away from the suture threads 4, allowing the threads to cool and solidify together, all the while being held in place by the compressible restraining pads 16. For those embodiments of the invention that include an integral pressure switch(s) 13c, to regulate energy delivery, the energy can be turned off when the pressure drops below a certain predetermined threshold as operator relieves the pressure, indicated by the turning off of indicator lights 13d and 13e, or the operator can manually turn off the energy or a timer could be employed as well. The turning on and off of power to the fusing pad 13 can be indicated with a light 13d, and ideal fusing pressure, from the pressure switch(s) 13c can be indicated with a light 13e, such switches connected to the fusing pad 13 and the pressure switch(s) 13c by reporting conduits 13f. In some preferred embodiments an electronic controller is used to control the indicator lights, in response to sensor and actuator inputs, but is not illustrated for diagrammatical clarity. While some embodiments of the invention use electricity and electrical heating elements, such as resistive and ceramic, all well known to the art, other embodiments of the invention use lasers and optical fibres (energy/chemical conduit 13a) to deliver photonic heat energy to the fusing pad 13, which in this case could be distal end of the optical fibre or a lens to which the fibre is attached. Those preferred embodiments that use ceramic heating elements for the fusing pad 13, can also be used to cool the fused area 13b by methods well known to the art, including switching the polarity of the direct electrical current. While the illustrations indicate a remote source of power, other embodiments of the invention use a battery which is integral to the unit. Other embodiments of the invention deliver polymer solvents along the energy/chemical conduit 3a. which would be tubes, rather than wires, to the fusing pad 13, while others may deliver adhesives. All of these embodiments of the invention are within the ambit of the invention. Various embodiments of the invention have different shapes for the various pads, for example, FIG. 5d illustrates a circular compressible restraining pad 16 surrounding a circular fusing pad 13, on the clamping end 9. It should be noted that one or more fusing pad may be used, for example some embodiments of the invention place one on clamping end 9a as well as on clamping end 9. Also, the method of collecting together the suture threads 4 can vary depending upon the circumstances of their use. As noted above the example illustrated in FIG. 10a places the suture threads 4 together in a recess 20; however, the embodiment illustrated in FIG. 5a puts them together in adjoining recesses 20, abutted by guides 15; while the embodiment illustrated in FIGS. 5b, 5c and 5d are on flat surfaces. These examples are meant to be only examples of many embodiments that have different means of collecting and positioning the suture threads 4 together for fusing, but all of which come within the ambit of the invention.

FIGS. 5e to 5h illustrate a sequence of steps that might be used to gather together the threads 4 for fusing together. In this embodiment of the invention a springy clamping end 9a, in combination with a projection 9b, can grip thread 4, so that another thread may then be gathered together with the first thread 4, so they may be fused together. FIG. 5e illustrates how the first thread 4 is positioned above the bottom groove 20 in springy clamping end 9a. FIG. 9f illustrates how the projection 9b on springy clamping end 9a, when in contact with the upper clamping end 9 causes the springy clamping end 9a to deflect under load, causing the groove 20 to open to accommodate the thread 4. FIG. 5g illustrates how the groove 20 on the springy clamping end 9a closes and grips the thread 4 when the clamping ends 9, 9a are then separated and the springy clamping end 9a unloads. As illustrated in FIG. 5h, the operator may then collect the second thread 4 and position it in the upper groove 20 in the upper clamping end 9 and then bring the two threads 4 together in preparation for fusing them together. It is to be understood that this method of gathering the threads 4 together are examples of the embodiments of the invention. The number of grooves 20, the position and number of guides 15 may vary, depending upon the circumstances of use. It is to be noted that the upper clamping end 9 may also be springy, allowing for more effective gripping of the upper thread 4 as well. The projection 9b may also be positioned such that the forceps can be used to manipulate a suture or other object, normally larger in diameter than the thread 4. It should be noted that these features may be incorporated into forceps and end-effectors, such as those illustrated in FIGS. 14 and 15.

FIG. 5i and 5j illustrate an alternative method of loading and unloading the springy clamping end 9a to gather and release the suture thread 4. This more direct method relies on a finger hole 8c and draw wire 8f, which is attached to the springy clamping end 9a at the point of attachment 8g. When the finger hole 8c and draw wire 8f are drawn 8h by the operator, as illustrated in FIG. 5j, along guides 8d and 8e, the springy clamping end 9a bends, enlarging the groove 20 in clamping end 9a. When the operator releases the drawing force 8h, the springy clamping end 9a unloads, reassuming its original orientation, which firmly grips the suture thread 4. While the embodiment illustrated in FIG. 5i and 5j uses a finger hole 8c and is manually operated, it is to be understood that the drawing mechanism and its operation may be affected by any means, including robotic means, well known to the art and all are within the ambit of the invention. It is also to be understood that both clamping ends 9 and 9a, or 9 alone might be springy and controlled by similar means.

FIG. 10b, 10c and 10d illustrate an embodiment of the invention which has a curved clamping end 9, but is otherwise the same as those embodiments described in FIGS. 10 and 10a. This is to illustrate examples of the many shapes that the invention may assume, in addition to the scissor and tweezers type, and still be within the ambit of the invention. The embodiments of the invention may of course assume other shapes, as are convenient for the particular purpose to which they are applied, and all be within the ambit of the invention. FIG. 10d also illustrates the inclusion of a cutting element 23, which allows the surgeon to cut the suture thread, or other body, during the procedure. This or these cutting elements 23 may be placed at any convenient place on the forceps or any of the embodiments described herein, such as on end-effector 7e illustrated in FIG. 15, and all are within the ambit of the invention.

As mentioned above, the forceps can be imparted with back and forth motion as well as freedom to move in other directions, including rotary. FIG. 11 and 11a, illustrate how a embodiment of the invention 7b can move laterally 11e in response to the operator moving his fingers, such that the upper and lower distal arms 11, 11a move laterally with respect to one another. This is possible as the bushing 10e, illustrated in FIG. 11 can distort 10f in direction 11f as illustrated in FIG. 11a. It is important to note that this embodiment of the invention can move in any direction in the horizontal plain, imparting different rotations and directions to anything that might be in its grasp.

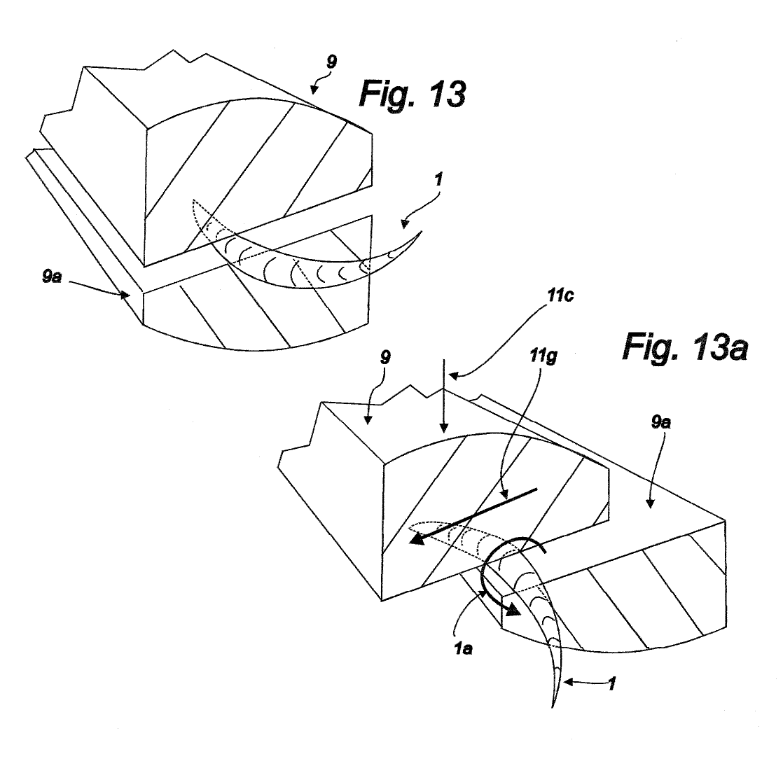

FIGS. 12 and 12a illustrate how this lateral motion 11e can control the attitude of the suture needle 1 which is grasped at the distal ends of the clamping end 9, 9a, moving the suture needle 1 in arc 1a, as illustrated in FIG. 12a.

FIGS. 13 and 13a illustrate how some embodiments of the invention impart motion to gasped objects at the distal end of the clamping ends 9, 9a when the mating surfaces are on the bias, rather than horizontal. For example, if one or both of the forceps arms 11, 11b are springy in a horizontal plane, as the arm is pressed down 11c, the sloping surfaces of clamping ends 9, 9a will cause the flexible upper distal arm 11 to bend horizontally in direction 11g. If the forceps in FIG. 11, 11a had biased clamping ends 9, 9a rather than horizontal ones, the operator would have a much easier time imparting a lateral motion 11e on the forceps, as the sloping surfaces would assist him imparting the desired motion, rather than relying solely on his laterally moving the upper distal arm 11 laterally with lateral motions of his thumb and fingers only.

Those embodiments of the invention have an arced and springy upper distal arm 11, as illustrated in FIG. 14, 14a, and this is combined with biased mating surfaces, such as those illustrated in FIGS. 13 and 13a, lateral 11e as well as longitudinal motions 11d are possible. Depending upon the relative horizontal and vertical springiness of the upper distal arm 11, the forceps can be engineered to first move longitudinally 11d, and then laterally 11e or vise versa, Other preferred embodiments might build the horizontal springiness into the lower distal arm 11, or some other combination thereof.

While the examples of the invention have referred to their medical use, it should be understood that it is not limited to these uses, for example the forceps may be used for many industrial purposes and recreational purposes, such as industrial robots and tools used to sew materials together as well as knot or polymer line fusing to attach fishing lures and hooks on fishing line, to name a few. Any reference to suture needle should be read as to include any object that might be held between the clamping ends 9 and 9a.

While reference has been made to features of the upper and lower distal arm 11, 11a, it should be noted that these features could be applied to both, likewise the position and number of features in the examples given may be vary depending upon the circumstances of their use, and all are within the ambit of the invention.

Many examples of the invention have been disclosed herein, however it is to be understood that the examples and illustrations have included many features which can be applied to each example given, in various combinations, and all are within the ambit of the invention.

While the present invention has been described in conjunction with preferred embodiments, it is to be understood that modifications and variations may be resorted to without departing from the spirit and scope of the invention as those skilled in the art will readily understand. Such modifications and variations are considered to be within the purview and scope of the inventions and appended claims.

What is claimed is:

1. A system comprised of a straight or curved needle or member, having opposed points at each end, and
having intermediate to the said points, a point(s) of attachment, detachable attachment, connection or slidable connection(s), which
anchors the proximal end of thread or other flexible filamentous material.

2. The system in claim 1, in which the said point of attachment may be comprised of a bonding surface, or a blind or open hole, depression, socket or slot onto or into which
a closed thread loop is formed, or onto or into which
the proximal end of the threat is fed, swaged or bonded, or into which
the proximal end of the thread, having one or more balls, disks or enlargements, is constrained or grasped by the complementary shape or restricting geometry of the said hole, socket or slot, such that
the ball or enlargement may move and/or rotate, in or about the grasping or restricting socket, hole or depression and/or move along the slot, while still being connected to the said needle.

3. The system in claim 1, in which two opposed, tapered, and shallowing, grooves are formed into the needle or member, adjacent to the said point of attachment, and
each said groove runs in opposite directions from the said point of attachment, and parallel to the longitudinal axis of the needle, such that
either groove accommodates the proximal end of thread as the thread is pressed into one of the grooves, when
the said needle or member, and attached thread are passed into a body,
so as to streamline the thread against the needle or member, and minimize friction between them and the body into which they are passed.

4. The system in claim 1, and a method, in which either point of such needle or member, with connected thread, may be passed sequentially into and out of a body forming stitches, and
the operator may choose to alternate, or sequence, which of the two points he introduces into such body on the initiation of each succeeding stitch, and
the operator may by causing the clamping ends of the forceps to slide laterally with respect to each other, thereby rotate the needle or member and thus select the orientation of the needle as it pierces, enters the body and is withdrawn from the body.

5. The system in claim 1, and method by which the operator can apply a mattress stitch, helical stitch, or other stitches with one hand manually or one end-effector remotely or robotically, and without switching the forceps to another hand or end-effector.

6. A system and method comprised of forceps or end effectors (hereafter each referred to as “forceps”) having clamping ends which,
are connected by a sliding, flexible, springy or compliant connection or member, which
can grasp the needle or member, and
while grasping the needle or member, said clamping ends can move laterally with respect to one another, in response to an input from the operator, and
thereby cause the said needle or member to rotate about various selected exes, including the longitudinal axis of the needle or member, at the points on which the needle is grasped.

7. The system and method of claim 6, in which the forceps arms, to which the clamping ends are attached, are slideably connected, and/or compliantly connected with deformable elements such that the operator can close the clamping ends to grasp a needle or member, and
then once grasped, can move the forceps arms laterally with respect to each other, and thereby cause the clamping ends to move laterally with respect to each other, which causes the needle or member which is grasped to rotate in various selected axes.

8. The system and method in claim 6, in which one of the forceps ends are arched and flexible, and the other forceps end is relatively rigid, and
the arched and flexible forceps end is sufficiently stiff that sufficient force can be transferred from the operator's force input to the clamping ends to grasp and hold the needle or member, and
having grasped the said needle or member, the arched and flexible forceps end is sufficiently flexible that upon the application by the operator of a greater force in approximately the same direction, the arched and flexible forceps end flattens and extends,
thereby causing the clamping ends to move laterally with respect to each other,
thereby causing the needle or member grasped between the said clamping ends to rotate in various selected axes.

9. The system and method of claim 6 in which the operator's inputs are applied manually by the operator's finger(s) and thumb(s) in finger holes or finger and hole recesses, or remotely by actuators, wires and other mechanical and/or electrical means.

10. The system and method of claim 6 in which the clamping surfaces of the clamping ends can be approximately parallel with the longitudinal axes of the forceps arms, or be approximately normal or at various other angles, to the said longitudinal axes of the said forceps arms.

11. The system and method of claim 6 in which
one or more grooves may be incorporated into one of the clamping ends, to cradle part of the needle or member, and
allow the needle to rotate in the said groove, but
prevent the said needle or member from walking or rolling down the clamping surface, in which the groove is formed,
while the clamping ends, both in contact with the said needle or member, move laterally relative to each other.

12. The system and method of claim 6 in which one or more grooves are formed in one the clamping surface(s) having a relatively flexible clamping end, and
in response to the operators force input, the flexible clamping end bends, when said flexible clamping end contacts a bump or protrusion on the mating surface of the other clamping end, and
the said groove opening enlarges, thereby allowing a suture thread or other member to lie in the groove, and
when the said force is released, the groove closes around the said suture thread or member, restraining it.

13. A system or device in claim 6, in which scissor type, chop type or other types of blade are incorporated into the arms of the forceps, which can be used to cut the suture thread.

14. A system or device in claim 6 in which the clamping end working surfaces, which mate on closing contact, are not normal to the clamping motion of the forceps, and
the clamping motion of the forceps causes the working surfaces of the clamping ends to slide laterally with respect to each other, and
thereby rotate a needle or other member which is grasped between the working surfaces of the forceps.

15. A system and method in claim 6, in which the clamping ends and/or distal arms of the forceps include guides to gather together the threads for welding or fusing.

16. A system and method comprised of forceps having clamping ends, one or both of which includes a fusing pad which is heated or exudes chemical(s) upon the input from the operator, and which
fuses or welds threads grasped together between the said clamping ends, and
the said threads are made of plastic, monofilament or other thermally and/or chemically fusible material, and
the said fused or welded thread(s) are then released from the clamping ends.

17. A system and method in claim 16 and method comprised of forceps having clamping ends, one or both of which includes a fusing pad which is first heated by the delivery of energy to the pad, upon the input from the operator,
followed by deactivation of the energy, which fuses or welds together thread segments, held between the said clamping ends, and
the fused or welded threads are then released from the clamping ends,
the said threads are made of plastic, monofilament or other thermally fusible material.

18. A system and method in claim 16 comprised of forceps having clamping ends, one or both of which include pads which extrude adhesive or solvent(s) or welding chemical on the input of the operator, and
such adhesive or solvent is delivered through a conduit, to the pad(s), and
fuse or weld together thread segments held between the said clamping ends, and
the said threads are made of plastic, monofilament or chemically fusible material.

19. The system and claim 16 in which the fusing pad is surrounded by a compressible restraining pad which constrains the thread(s) to be fused or welded together before, during and after the fusing pad, or the chemical exuded by the said pad has made contact with the said threads, but releases the said fused or welded thread(s) when the operator, manually or remotely, further separates the clamping ends.

20. The system in claim 16 in which sensors detect the temperature and/or pressure of the fusing pad and/or the thread(s) at the point of the fuse or weld, and signal a controller or computer which either signals the operator of the pressure and temperature information at each step of the welding or fusing process, or automatically controls the application of temperature or pressure or both.

21. The system and method in claim 16 for heat or chemically fusing or welding joins in place of knots for surgery, to attach fishing lures and for all other purposes.

22. The system of device in claim 16 which includes controls for heat and temperature, including foot controls.
